(19) United States
(12) Patent Application Publication
(10) Pub. No.: US 2009/0325904 A1
(43) Pub. Date: Dec. 31, 2009

(54) MALEIC ACID MONOSALT OF ANTIVIRAL AGENT AND PHARMACEUTICAL COMPOSITION CONTAINING THE SAME

(75) Inventors: Ji Hye Lee, Daejeon (KR); Ki Sook Park, Daejeon (KR); Jung Min Yun, Daejeon (KR)

Correspondence Address: BIRCH STEWART KOLASCH & BIRCH, PO BOX 747, FALLS CHURCH, VA, 22040-0747, US

(73) Assignee: LG Life Sciences Ltd., Seoul (KR)

(21) Appl. No.: 12/522,046

(22) PCT Filed: Jan. 11, 2008
(86) PCT No.: PCT/KR08/00194

§ 371 Date: Aug. 10, 2009

(30) Foreign Application Priority Data
Jan. 17, 2007 (KR) 10-2007-0005269

(51) Int. Cl. A61K 31/675; C07D 473/02; A61P 31/12

(52) U.S. Cl. 514/81; 544/277

(57) ABSTRACT

The present invention relates to 3-[({1-[(2-amino-9H-purin-9-yl)methyl]cyclopropyl}oxy)methyl]-8,8-dimethyl-3,7-dioxo-2,4,6-trioxa-3λ5-phosphanon-1-yl-pivalate maleic acid monosalt, and pharmaceutical composition containing the same.

### TECHNICAL FIELD

The present invention relates to 3-[({1-[(2-amino-9H-purin-9-yl)methyl]cyclopropyl}oxy)methyl]-8,8-dimethyl-3,7-dioxo-2,4,6-trioxa-3λ5-phosphanon-1-yl-pivalate maleic acid monosalt of the following formula (1), and pharmaceutical composition containing the same:

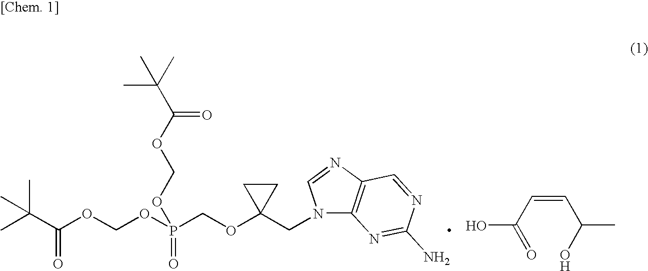

### BACKGROUND ART

The free base corresponding to the above compound of formula (1), i.e., the compound which is not combined with an acid, is a new antiviral compound that was disclosed in Korean Patent No. 0441638 and WO02/057288. This free base is currently undergoing clinical study. It has a potent antiviral effect, particularly against the Hepatitis B Virus (HBV) and the Human Immunodeficiency Virus (HIV). However, this free base is unstable under heat and moisture, which poses problems when developing the compound as a pharmaceutical drug product.

### DISCLOSURE OF THE INVENTION

The present inventors have researched various ways to resolve the problems with the free base. As a reset of their research, they have discovered that the maleic acid monosalt of formula (I) of this invention can have a crystalline characteristic and excellent solubility, is non-hygroscopic, and is highly stable under heat.

Thus, the purpose of the present invention is to provide the maleic acid monosalt of formula (1).

The present invention further provides a pharmaceutical composition comprising the maleic acid monosalt of formula (1) as an active ingredient.

### BRIEF DESCRIPTION OF THE DRAWINGS

FIG. 1 shows the powder X-ray diffraction pattern of one embodiment of 3-[({1-[(2-amino-9H-purin-9-yl)methyl]cyclopropyl}oxy)methyl]-8,8-dimethyl-3,7-di oxo-2,4,6-trioxa-3λ5-phosphanon-1-yl-pivalate maleic acid monosalt of the present invention.

FIG. 2 shows the result from differential scanning calorimetry of one embodiment of 3-[({1-[(2-amino-9H-purin-9-yl)methyl]cyclopropyl}oxy)methyl]-8,8-dimethyl-3,7-di oxo-2,4,6-trioxa-3λ5-phosphanon-1-yl-pivalate maleic acid monosalt of the present invention.

FIG. 3 shows the content (%) change over time and temperature of 3-[({1-[(2-amino-9H-purin-9-yl)methyl]cyclopropyl}oxy)methyl]-8,8-dimethyl-3,7-di oxo-2,4,6-trioxa-3λ5-phosphanon-1-yl-pivalate free base and one embodiment of its maleic acid monosalt.

FIG. 4 shows the in-vitro activity and cytotoxicity result against hepatitis B virus of 3-[({1-[(2-amino-9H-purin-9-yl)methyl]cyclopropyl}oxy)methyl]-8,8-dimethyl-3,7-di oxo-2,4,6-trioxa-3λ5-phosphanon-1-yl-pivalate free base and one embodiment of its maleic acid monosalt.

### BEST MODE FOR CARRYING OUT THE INVENTION

The present invention provides 3-[({1-[(2-amino-9H-purin-9-yl)methyl]cyclopropyl}oxy)methyl]-8,8-dimethyl-3,7-dioxo-2,4,6-trioxa-3λ5-phosphanon-1-yl-pivalate maleic acid monosalt of the following formula (1):

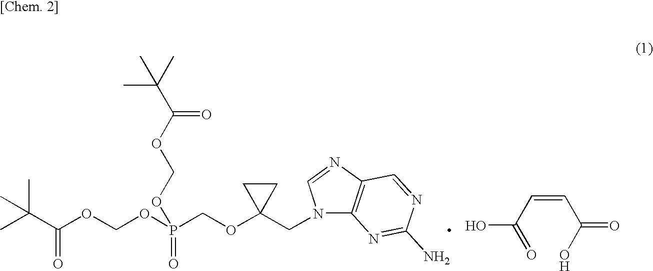

Unless otherwise indicated in the present specification, the term “maleic acid monosalt of formula (1)” means a salt wherein 1 eq of the corresponding free base [i.e., the free base of maleic acid monosalt of formula (1)] is combined with 0.7 to 1.3 eq, preferably 0.9 to 1.1 eq, more preferably 1 eq of maleic acid.

The maleic acid monosalt of formula (1) can be prepared by a process which comprises a step of mixing the free base and maleic acid with an organic solvent, which is a process that is wel known in the art (see Pharmaceutical Salts, Journal of Pharmaceutical Sciences, Donald C. Monkhouse et al, 1, 66(1), 1977 and Salt selection for basic drugs, International Journal of Pharmaceutics, Philip L. Gould, 201, 33, 1986).

Specifically, maleic acid monosalt of formula (1) can be prepared by dissolving the free base in an organic solvent in the ratio of from 50 to 1,000 mg of the free base per ml solvent, adding (preferably, in drops) maleic acid of the below mentioned amount thereto, and stirring to produce a solid. The organic solvent may be selected without restriction from the conventional organic solvents that can be used for forming a salt, but preferably selected from the group consisting of ethyl acetate, butyl acetate, acetonitrile, chloroform, acetone, methanol, ethanol, propanol, isopropanol, tetrahydrofuran, methyl ethyl ketone, isopropyl acetate, dioxane, n-hexane, cyclohexane, di-ethylether, t-butylether and mixtures thereof. The amount of maleic acid to be added is not limited to a particular amount, but preferably the amount is 0.7 to 1.3 eq, more preferably 0.9 to 1.2 eq, and most preferably 1.0 to 1.1 eq with respect to 1 eq of the free base. The resulting sold undergoes the conventional work-up processes such as filtration, washing, drying, etc.

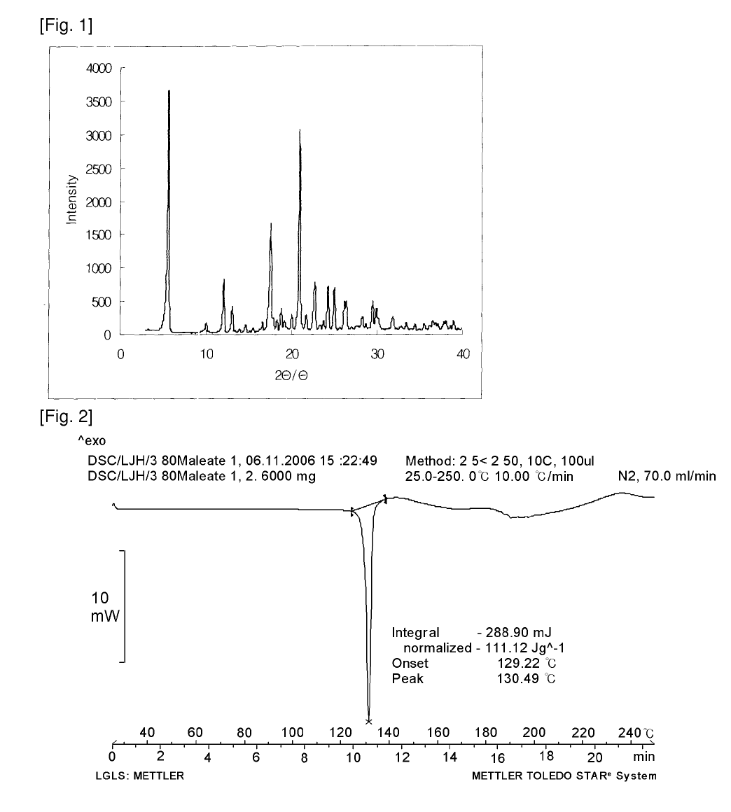

The maleic acid monosalt of formula (1) prepared by the above process is preferably obtained as a crystalline sold. That is, the maleic acid monosalt of the present invention can have a characteristic crystalline structure showing significant peaks at 2Θ=5.6, 12.1, 17.5 and 20.9° (2Θ, +/−0.2) in the powder X-ray diffraction pattern. More preferably, the maleic acid monosalt has the crystalline structure showing characteristic peaks at 2H)=5.6, 10.0, 12.1, 13.1, 17.5, 18.8, 20.9, 22.8, 24.3, 25.1 and 26.5° (2Θ, +/−0.2) in the powder X-ray diffraction pattern (see FIG. 1). This crystal form shows a melting point endothermal onset peak at 129° C. in the differential scanning calorimetry (10° C./min) (see FIG. 2).

The maleic acid monosalt of formula (1) is non-hygroscopic, and has better solubility and better stability under heat and moisture than the corresponding free base or other salts thereof. It is also in the form of a crystalline solid. Therefore, the physico-chemical properties of the maleic acid monosalt of formula (1) make it suitable to be developed as a pharmaceutical drug product.

As explained more in detail in the following Experiments, the free base developed as an antiviral agent is highly unstable under heat and moisture, and thus, it is difficult to be used as a raw material for pharmaceutical drug product. Accordingly, there was difficulty in developing the free base as a drug substance. The present inventors tried to resolve the problems with the free base by preparing several kinds of pharmaceutically acceptable salts. During the preparations, it was discovered that some of the salts could not easily be obtained as a crystalline solid. The present inventors succeeded in obtaining salts with maleic acid, p-toluenesulfonic acid, methanesulfonic acid, naphthalenesulfonic acid, or ethanesulfonic acid as crystalene solids. The inventors performed thermal stability test at stressed condition for the free base and several salts obtained as crystalline solids. The tests showed that the free base and the salts except the maleic acid monosalt are very unstable under heat. The maleic acid monosalt remained almost intact without decomposition for up to 8 weeks under the high temperature of 60° C., whereas the free base decomposed entirely with only about 1% remaining after 8 weeks. The other crystalline salts almost decomposed within 2 weeks. Thus, the maleic acid monosalt of the present invention exhibits superior heat-stability compared to the free base or other organic salts. Further, it was not easy to obtain crystalline solids from the other salts, but the crystalline solid of the maleic acid monosalt could easily be obtained according to the above process. That is, the maleic acid monosalt could be readily applied to production on an industrial scale.

The maleic acid monosalt of the present invention also exhibits improved solubility depending on the levels of pH. Specifically, the free base shows high solubility of 36 mg/ml or more at a low pH of 2 or less, but the solubility drastically decreases as the pH increases, i.e., a solubility of 1 mg/ml or less at pH 6 or more. Due to such characteristics, the free base is entirely dissolved and absorbed in the stomach, but there is the risk that the compound can precipitate out as it travels to the internal organs which have a higher pH level. However, the maleic acid monosalt of the present invention exhibits relatively constant solubility of about 7 to 3 mg/ml at the pH range of 2 to 6.5. In fact, the solubility of the maleic acid monosalt at pH 6.5 is three times higher than the free base. It suggests that, in the aspect of medicinal efficacy, the maleic acid monosalt will be absorbed more into the body, and the risk of precipitation after absorption can be excluded even with the pH change. That is, the maleic acid monosalt of the present invention exhibits superior solubility even at different pH levels to the free base.

Based on the above physical, physiological properties, there are great advantages in using the maleic acid monosalt of the present invention for the prevention or treatment of viral infections. Thus, the present invention provides a pharmaceutical composition for the prevention or treatment of a viral infection, which comprises a therapeutically effective amount of the maleic acid monosalt of formula (1) and a pharmaceutically acceptable carrier. The virus to be most effectively treated by the present invention is from the group consisting of HBV and HIV.

Oral administration is the most preferable form of administration of the pharmaceutical composition comprising the maleic acid monosalt of formula (1) as the active ingredient, especially in a tablet or capsule.

The “therapeutically effective amount” of the maleic acid monosalt of formula (1) as an active ingredient varies with gender, age and diet of the subject patient, the severity of the disease to be treated, etc., and can be easily determined clinically by a skilled person in the art.

Korean Patent No. 0441638 and WO02/057288, each of which discloses the corresponding free base and effect thereof, can be referred to for the pharmacological effect, effective dose range, method of administration of the pharmaceutical composition comprising the maleic acid monosalt of formula (1) as an active ingredient.

The present invention is more specifically explained by the following examples and experiments which are intended to illustrate the present invention and in no way to limit the scope of the present invention.

HPLC Conditions

Contents of the free base of 3-[({1-[(2-amino-9H-purin-9-yl)methyl]cyclopropyl}oxy)methyl]-8,8-dimethyl-3,7-dioxo-2,4,6-trioxa-3λ5-phosphanon-1-yl-pivalate and salts thereof were measured by high performance liquid chromatography (HPLC). The specific measuring conditions are fisted below:

Column: Waters Symmetry Shield C18 (4.6×250 mm, 5 μm)

Column Temperature: 30° C.

Flow rate: 1.0 ml/min

Detection Wavelength: UV 309 nm

Eluents: A. Tetrahydrofuran/Water=3/7

B. Tetrahydrofuran/Water=8/2 (v/v, gradient elution)

Mixing ratio of the eluents over time

| Time (min) | Eluent A | Eluent B |
|---|---|---|
| 0 | 100 | 0 |
| 16 | 100 | 0 |
| 30 | 0 | 100 |
| 32 | 0 | 100 |
| 34 | 100 | 0 |
| 45 | 100 | 0 |

Conditions for Differential Scanning Calorimetry

DSC curve was obtained with Mettler-Toledo DSC821 system. The thermal behavior was studied by heating 2-5 mg of sample in an aluminium sample pan under nitrogen gas flow over the temperature range 25-250° C. at heating rate of 10° C./min. The sample pan cover had a pin-hole to avoid pressure build-up inside the sample pan.

Conditions for X-Ray Diffraction

The sample (about 20 mg) was packed on a sample holder, which was then put into a Philips x-ray generator (PW1710). The diffraction pattern of the sample was attained in the range of 3˜40°/2θ. Details of the analysis conditions are fisted below:

Time per step: 0.5

Stepsize: 0.03

Scan Mode: step

Voltage/Current: 40 kV/30 mA

2 θ/θ Reflection

Cu-target (N-filter)

Source Slit: 1.0 mm

Detector Slits: 0.15 mm, 1.0 mm

### Comparative Example 1

### Free Base of 3-[({1-[(2-amino-9H-purin-9-yl)methyl]cyclopropyl}oxy)methyl]-8,8-dimethyl-3,7-dioxo-2,4,6-trioxa-3λ5-phosphanon-1-yl-pivalate

The title compound was prepared according to the process described in Korean Patent No. 0441638 and WO02/057288.

### Example

3-[({1-[(2-amino-9H-purin-9-yl)methyl]cyclopropyl}oxy)methyl]-8,8-dimethyl-3,7-dioxo-2,4,6-trioxa-3λ5-phosphanon-1-yl-pivalate maleic acid monosalt

The free base obtained in Comparative Example 1 (100 mg) was dissolved in ethyl acetate (1 ml). Maleic acid (1 eq) was added, and the mixture was stirred for 1 h to produce a solid. The resulting sold was filtered, washed with ethyl acetate, and dried to yield 111.4 mg (Yield 91.3%) of the maleic acid monosalt as a crystalline sold.

Content: 99.3%

Differential Scanning Calorimetry: 129° C. (Endothermic: 111 J/g)

¹H NMR (CD₃OD): δ 8.64 (s, 1H), 8.35 (s, 1H), 6.30 (s, 2H), 5.62 (m, 4H), 4.37 (s, 2H), 4.17 (d, 2H), 1.20 (s, 18H), 0.99 (m, 4H)

Powder X-ray Diffraction Spectrum: 2Θ=5.6, 10.0, 12.1, 13.1, 17.5, 18.8, 20.9, 22.8, 24.3, 25.1 and 26.5° (20, +/−0.2)

### Comparative Example 2

### 3-[({1-[(2-amino-9H-purin-9-yl)methyl]cyclopropyl}oxy)methyl]-8,8-dimethyl-3,7-dioxo-2,4,6-trioxa-3λ5-phosphanon-1-yl-pivalate maleic acid trisalt

The free base obtained in Comparative Example 1 (5 g) was dissolved in ethyl acetate (50 ml). Maleic acid (3 eq) was added. The mixture was stirred for 12 h, and n-hexane (20 ml) was added thereto to produce a solid. The resulting solid was filtered, washed with n-hexane, and dried to yield 6.52 g (Yield 78.6%) of the maleic acid trisalt.

Content: 98.7%

¹H NMR (CD₃OD): δ 8.70 (s, 1H), 8.46 (s, 1H), 6.31 (s, 6H), 5.62 (m, 4H), 4.38 (s, 2H), 4.17 (d, 2H), 1.20 (s, 18H), 0.99 (m, 4H)

### Comparative Example 3

### 3-[({1-[(2-amino-9H-purin-9-yl)methyl]cyclopropyl}oxy)methyl]-8,8-dimethyl-3,7-dioxo-2,4,6-trioxa-3λ5-phosphanon-1-yl-pivalate p-toluenesulfonic acid monosalt

The free base obtained in Comparative Example 1 (100 mg) was dissolved in ethyl acetate (1 ml). p-Toluenesulfonic acid (1 eq) was added, and the mixture was stirred for 1 h to produce a sold. The resulting solid was filtered, washed with ethyl acetate, and dried to yield 106.4 mg (Yield 78.2%) of the p-toluenesulfonic acid monosalt.

Content: 99.43%

¹H NMR (CD₃OD): δ 8.74 (s, 1H), 8.57 (s, 1H), 7.68 (d, 2H), 7.20 (d, 2H), 5.59 (m, 4H), 4.37 (s, 2H), 4.14 (d, 2H), 2.34 (s, 3H), 1.13 (s, 18H), 0.98 (m, 4H)

### Comparative Example 4

### 3-[({1-[(2-amino-9H-purin-9-yl)methyl]cyclopropyl}oxy)methyl]-8,8-dimethyl-3,7-dioxo-2,4,6-trioxa-3λ5-phosphanon-1-yl-pivalate p-toluenesulfonic acid disalt

The free base obtained in Comparative Example 1 (5 g) was dissolved in ethyl acetate (50 ml). p-Toluenesulfonic acid (2 eq) was added, and the mixture was stirred for 1 h to produce a sold. The resulting solid was filtered, washed with ethyl acetate, and dried to yield 7.01 g (Yield 81.5%) of the p-toluenesulfonic acid disalt.

Content: 97.8%

¹H NMR (CD₃OD): δ 8.77 (s, 1H), 8.61 (s, 1H), 7.71 (d, 4H), 7.23 (d, 4H), 5.62 (m, 4H), 4.40 (s, 2H), 4.17 (d, 2H), 2.37 (s, 6H), 1.20 (s, 18H), 0.99 (m, 4H)

### Comparative Example 5

### 3-[({1-[(2-amino-9H-purin-9-yl)methyl]cyclopropyl}oxy)methyl]-8,8-dimethyl-3,7-dioxo-2,4,6-trioxa-3λ5-phosphanon-1-yl-pivalate methanesulfonic acid monosalt

The free base obtained in Comparative Example 1 (100 mg) was dissolved in ethyl acetate (1 ml). Methanesulfonic acid (1 eq) was added in drops, and the mixture was stirred for 1 h to produce a solid. The resulting solid was filtered, washed with ethyl acetate, and dried to yield 95.2 mg (Yield 80.6%) of the methanesulfonic acid monosalt.

Content: 97.6%

¹H NMR (CD₃OD): δ 8.79 (s, 1H), 8.58 (s, 1H), 5.60 (m, 4H), 4.38 (s, 2H), 4.14 (d, 2H), 2.70 (s, 3H), 1.17 (s, 18H), 1.01 (m, 4H)

### Comparative Example 6

### 3-[({1-[(2-amino-9H-purin-9-yl)methyl]cyclopropyl}oxy)methyl]-8,8-dimethyl-3,7-dioxo-2,4,6-trioxa-3λ5-phosphanon-1-yl-pivalate naphthalenesulfonic acid monosalt

The free base obtained in Comparative Example 1 (5 g) was dissolved in ethyl acetate (30 ml). Naphthalenesulfonic acid (1 eq, 1.97 g) was dissolved in water (5 ml), which was then added in drops. After stirring the mixture for 15 h, the solvent was thoroughly removed under reduced pressure. Ethanol and diethylether were added to the residue to precipitate a white crystal. The resulting solid was filtered, washed with a solvent mixture of ethanol and diethylether, and dried to yield 6.2 g (Yield 90.0%) of the naphthalenesulfonic acid monosalt.

Content: 91.4%

¹H NMR (CD₃OD): δ 8.48 (s, 2H), 8.44 (s, 1H), 7.95 (d, 1H), 7.83 (m, 3H), 7.50 (m, 2H), 5.63 (m, 4H), 4.23 (s, 2H), 3.95 (d, 2H), 1.18 (s, 18H), 1.01 (m, 4H)

### Comparative Example 7

### 3-[({1-[(2-amino-9H-purin-9-yl)methyl]cyclopropyl}oxy)methyl]-8,8-dimethyl-3,7-dioxo-2,4,6-trioxa-3λ5-phosphanon-1-yl-pivalate ethanesulfonic acid monosalt

The free base obtained in Comparative Example 1 (5 g) was dissolved in ethyl acetate (30 ml). Ethanesulfonic acid (1 eq, 1.05 g) was added thereto and thoroughly dissolved. After stirring the mixture for 1 h, the solvent was thoroughly removed under reduced pressure. Ethanol, diethylether and n-hexane were added to the residue to precipitate a white crystal. The resulting sold was filtered, washed with a solvent mixture of ethanol and diethylether, and dried to yield 5.0 g (Yield 82.8%) of the ethanesulfonic acid monosalt.

Content: 90.0%

¹H NMR (CDCl₃): δ 8.60 (s, 1H), 8.51 (s, 1H), 5.63 (m, 4H), 4.32 (s, 2H), 4.00 (d, 2H), 2.92 (m, 2H), 1.29 (m, 3H), 1.19 (s, 18H), 1.01 (m, 4H)

Experiment 1: Comparative Test 1 for the Stability Under Heat and Moisture

30˜70 mg each of the maleic acid monosalt of the Example, the free base and the salts of Comparative Examples 1 to 5 was introduced into a glass vial, and stored under 40±2° C. and 75±5% RH. After 1, 4 and 8 weeks, 5 mg of each sample was taken, dissolved in a solvent mixture of tetrahydrofuran/water (1/1, v/v), and analyzed by HPLC. The results are summarized in the following Table 1.

TABLE 1
Stability test results for the maleic acid monosalt of formula (1),
its free base and the other salts under 40° C./75% RH
(residual content, %).

| Test Compound | | Week 1 | Week 4 | Week 8 |
|---|---|---|---|---|
| Example | Maleic acid monosalt | 99.4 | 99.3 | 98.3 |
| Comparative | Free base | 99.0 | 91.3 | 8.4 |
| Example 1 | | | | |
| Comparative | Maleic acid trisalt | 84.4 | 0.0 | 0.0 |
| Example 2 | | | | |
| Comparative | p-Toluenesulfonic | 96.5 | 71.0 | — |
| Example 3 | acid monosalt | | | |
| Comparative | p-Toluenesulfonic | 0.0 | 0.0 | 0.0 |
| Example 4 | acid disalt | | | |
| Comparative | Methanesulfonic acid | 50.7 | 0.0 | 0.0 |
| Example 5 | monosalt | | | |

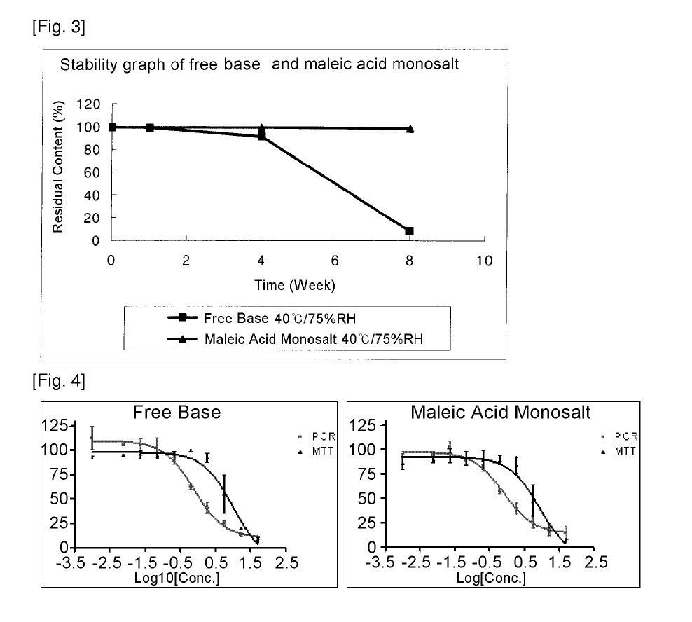

As seen from the results of Table 1, the maleic acid monosalt of formula (1) exhibits superior heat stability to the corresponding free base and the other salts. The stability results for the maleic acid monosalt and free base are depicted in FIG. 3.

Experiment 2: Comparative Test 2 for the Stability Under Heat and Moisture

About 5˜6 mg each of the maleic acid monosalt of Example, the free base and the salts of Comparative Examples 6 to 7 was introduced into a glass vial, and stored at a temperature of 60° C. After 1 or 2, 4 and 8 weeks, each sample in the glass vial was taken, dissolved in a solvent mixture of tetrahydrofuran/water (1/1, v/v), and analyzed by HPLC. The results are summarized in the following Table 2.

TABLE 2
Stability test results for the maleic acid monosalt of formula (1), its
free base and the other salts at 60° C. (residual content, %).

| | | 60° C. (about 4% RH) | | | |
|---|---|---|---|---|---|
| Test Compound | | Week 1 | Week 2 | Week 4 | Week 8 |
| Example | Maleic acid | 99.4 | — | 99.2 | 98.2 |
| | monosalt | | | | |
| Comparative | Free base | 98.2 | — | 79.2 | 1.2 |
| Example 1 | | | | | |
| Comparative | Naphthalenesulfonic | — | 74.7 | — | — |
| Example 6 | acid monosalt | | | | |
| Comparative | Ethanesulfonic acid | 0.4 | — | — | — |
| Example 7 | monosalt | | | | |

The results of Table 2 show that the maleic acid monosalt of formula (1) exhibits superior heat stability to the corresponding free base and the other salts under high temperature.

Experiment 3: Solubility Test at Various pH

5˜23 mg each of the maleic acid monosalt of the Example and the free base of Comparative Example 1 was placed into a glass bottle. 500 μl each of the various phosphate buffer solution and phosphoric acid solution having a specific pH value was added thereto. The glass bottle was placed in water to maintain a constant temperature of 25° C., and the mixture was stirred for 1.5 h. After filtration, the content in the filtrate was analyzed by HPLC, and the pH of the solution was measured. The measured pH values and the solubilities of the maleic acid monosalt and the free base are represented in the following Table 3.

TABLE 3
pH-Dependent solubility of the maleic acid monosalt of
formula (1) and the free base (mg/ml)

| | Example | Comparative Example 1 |
|---|---|---|
| Solution pH | (Maleic acid monosalt) | (Free base) |
| 2.0 | 5.6 | 36.5 |
| 3.2 | 7.0 | 8.3 |
| 4.0 | 4.2 | 1.7 |
| 6.5 | 2.9 | 0.8 |

Experiment 4: Pharmacological Effect and Cytotoxicity of the Maleic Acid Monosalt and Free Base

1) Cell Culture and Compound Treatment

The hepatitis B virus-producing cell line, HepG_{2.2.15} (M. A. Shels, et al., Proc. Natl. Acad. Sci. USA 84, 1005 (1987)), was cultured in DMEM (Dulbecco's Modified Eagle Media; Life Technologies) containing 10% FBS (Fetal Bovine Serum), 1% ABAM (Antibiotic-Antimycotic) and Geneticin whose final concentration was measured as 400 μg/ml. The cells were cultured to confluency, treated with trypsin, and distributed to 96 well microplate in a density of 2×10⁴ cells/well. After 24 h, the medium was changed and the compound treatment was carried out in intervals of 2 days by serially diluting the free base of Comparative Example 1 and the maleic acid monosalt of Example by three fold so that the final concentration was 50 μM to 8 nM in 200 μl of medium. Every test samples were duplicated. After 8 days from the first drug treatment, the culture medium was collected, and the cells were lysed by heating the cells to 100° C. for 10 min. In order to minimize the substances that interfere with the DNA amplification reaction, the culture medium was diluted by ten fold using water. The control group, cell culture medium which was not treated with the drug, was treated in the same manner as the above.

2) Pharmacological effect determination: quantitative analysis using real-time PCR reaction The culture medium (6 μl), which was pre-treated as the above, was added to polymerase/buffered solution mixture [10 mM Tris-HCl (pH 8.3), 50 mM KCl, 200 μM dNTP, 200 nM primiers, 200 nM probe, 3 mM MgCl₂, 1 unit AmplTaq DNA polymerase (Applied Biosystems, Foster City, Calif.)]. Using the real-time PCR machine (Rotor-gene 2000 Real-time Cycler: CORBETT Research.), 95° C. reaction was performed for 3 min, and then 95° C./20 sec-56° C./30 sec-85° C./20 sec reaction was repeated 45 times. The fluorecence was detected at 85° C. polymerization reaction.

5′-TCAGCTCTGTATCGGGAAGC-3′ and 5′-CACCCACCCAGGTAGCTAGA-3′ (Genotech) were used as 5′ primer and 3′ primer, respectively, and 5′-6-FAM-CCTCACCATACTGCACTCAGGCAA-BHQ-1-3′ (Proligo) was used as the fluorescence probe.

The automatically calculated amount of HBV DNA in the sample was analyzed by calculating the relative value of the subject sample with respect to the value of the sample untreated with the drug, and by using the statistical program PRISM (GraphPad Software, Inc.).

3) Cytotoxicity Determination

CC₅₀ value of the drug was determined by removing the medium, adding 100 μl of 0.1 mg/ml MTT (Thiazolyl Blue Tetrazolium Bromide: Sigma) to the residue, dyeing the residue for 2 h at 37° C., adding 100 μl of DMSO (Dimethyl Sulfoxide: Sigma), dissolving the resulting mixture by agitating for 2 h at room temperature, and measuring the absorbance at 540 nm.

EC₅₀ and CC₅₀ values for the free base of Comparative Example 1 and the maleic acid monosalt of the Example obtained from the above experiment are represented in the following Table 4.

TABLE 4

| Test Compound | EC₅₀ (μM) | CC₅₀ (μM) |
|---|---|---|
| Free base of Comparative | 1.1 ± 0.1 | 7.9 ± 3.0 |
| Example 1 | | |
| Maleic acid monosalt of | 1.2 ± 0.3 | 6.8 ± 2.4 |
| Example | | |

As can be seen from the results of Table 4, the in vitro test of intracellular pharmacological activity showed that both the free base of Comparative Example 1 and the maleic acid monosalt of Example exhibit similar activity (about 1 m M) and cytotoxicity (about 7 m M).

### INDUSTRIAL APPLICABILITY

3-[({1-[(2-amino-9H-purin-9-yl)methyl]cyclopropyl}oxy)methyl]-8,8-dimethyl-3,7-dioxo-2,4,6-trioxa-3λ5-phosphanon-1-yl-pivalate maleic acid monosalt of the present invention shows excellent stability under moisture and heat and maintains a constant solubility at different pH levels. Therefore, the present invention can maintain high quality of the active ingredient of the pharmaceutical composition for the prevention or treatment of viral infections, such as HBV or HIV infection, over a long period of time.

1. (3-[({1-[(2-amino-9H-purin-9-yl)methyl]cyclopropyl}oxy)methyl]-8,8-dimethyl-3,7-dioxo-2,4,6-trioxa-3λ5-phosphanon-1-yl-pivalate maleic acid monosalt.

2. The maleic acid monosalt of claim 1 in the form of crystalline solid.

3. The maleic acid monosalt of claim 2 having peaks at 20Θ=5.6, 12.1, 17.5 and 20.9° in its powder X-ray diffraction pattern.

4. The maleic acid monosalt of claim 3 having peaks at 2ΘH=5.6, 10.0, 12.1, 13.1, 17.5, 18.8, 20.9, 22.8, 24.3, 25.1 and 26.5° in its powder X-ray diffraction pattern.

5. Pharmaceutical composition for the prevention or treatment of viral infections, which comprises the maleic acid monosalt according to claim 1; and pharmaceutically acceptable carrier.

6. The composition of claim 5 wherein the virus is HBV.

7. The composition of claim 5 wherein the virus is HIV.

8. Pharmaceutical composition for the prevention or treatment of viral infections, which comprises the maleic acid monosalt according to claim 2; and pharmaceutically acceptable carrier.

9. Pharmaceutical composition for the prevention or treatment of viral infections, which comprises the maleic acid monosalt according to claim 3; and pharmaceutically acceptable carrier.

10. Pharmaceutical composition for the prevention or treatment of viral infections, which comprises the maleic acid monosalt according to claim 4; and pharmaceutically acceptable carrier.
